(19) United States
(12) Patent Application Publication
(10) Pub. No.: US 2026/0115417 A1
(43) Pub. Date: Apr. 30, 2026

(54) MULTI-SEGMENTED MICROCATHETER DEVICE

(71) Applicant: MicroLiner Technologies, Inc., Charlotte, NC (US)

(72) Inventor: Alexander Ryan Thomas, Charlotte, NC (US)

(21) Appl. No.: 19/151,767

(22) PCT Filed: Apr. 25, 2025
(86) PCT No.: PCT/US2025/026381

§ 371 Date: Jul. 29, 2025

Related U.S. Application Data
(60) Provisional application No. 63/638,516, filed on Apr. 25, 2024.

(51) Int. Cl. A61M 25/00; A61B 17/00; A61B 17/22; A61M 25/01; A61M 25/06; A61M 39/00; A61M 39/10

(52) U.S. Cl. CPC A61M 25/0043; A61B 17/00234; A61B 17/22; A61M 25/0021; A61M 25/0097; A61M 25/0102; A61M 25/0136; A61M 25/0662; A61M 39/10; A61B 2017/00238; A61B 2017/00305; A61B 2017/00367; A61B 2017/22038; A61M 2025/0042; A61M 2039/0009

(57) ABSTRACT

A multi-segmented microcatheter device includes a distal segment having an interior bore for a guidewire portion, a push-wire extension extending away from an end of the distal segment, a proximal segment having an interior lumen for a guidewire portion and a longitudinal channel to slidably engage the push-wire extension, a connector module to removably secure the proximal and distal segments together so a contiguous passage for a guidewire is formed from the interior bore and lumen and a motion lock hub that selectively locks the push-wire extension and the proximal segment together. Thereby, the proximal and distal segments can be manipulated in unison when they are secured together with the connector module and the push-wire extension and proximal segments are locked in a fixed relation through the

### CROSS-REFERENCE TO RELATED APPLICATIONS

This application claims priority to U.S. Provisional Patent Application No. 63/638,516 which was filed on Apr. 25, 2024, the entire contents of which is hereby incorporated by reference in its entirety.

### TECHNICAL FIELD

The present disclosure relates to the field of medical devices and methods. More particularly, this disclosure relates to devices and systems for atraumatic placement and use of a catheter and which involve microcatheter devices having multiple segments to enhance utility thereof.

### BACKGROUND

A catheter, as used in surgical procedures, is a tube inserted in a patient to perform a surgical procedure and/or place a prosthetic device, such as a coronary artery stent. Catheters can be particularly designed for cardiovascular, urological, gastrointestinal, neurovascular, and ophthalmic applications. The process of inserting a catheter is called “catheterization.”

One type of catheter, which is particularly useful for coronary procedures is a microcatheter, which is a small diameter catheter. During coronary procedures, particularly interventional ones, microcatheters are commonly used by physicians to access discrete regions of the arterial vasculature. Microcatheters are typically used to facilitate guidewire placement and exchange and can also be used to deliver therapeutic agents super selectively (i.e., into very distal or branch vessels). Indeed, microcatheters excel at navigating tortuous arterial anatomy and are widely used during percutaneous coronary as well as neurovascular interventions.

Microcatheters are routinely employed in complex endovascular procedures to support or exchange guidewires, access distal anatomy, cross lesions, deliver embolic agents, inject contrast media, and perform other tasks. For example, a microcatheter can be torqued in conjunction with a to assist passage across a lesion. However, a wide array of microcatheters—each tailored to a specific application—are commercially available. Indeed, key characteristics, such as overall length, often determine which device is suited for a given technique. For example, an Over-the-Wire (OTW) system typically requires a relatively long microcatheter (about 130-150 cm), whereas a rapid-exchange (RX) system employs a markedly shorter microcatheter (about 10-60 cm). Exchanging a short 180/190 cm guidewire for a long 300 cm guidewire can be labor and time intensive, because the first microcatheter must traverse the entire length of the guidewire before removal and a second microcatheter must then be advanced nearly the same distance to reach the lesion. Moreover, when only the microcatheter or guidewire needs to be exchanged, specialized methods-such as balloon-trapping—are required to maintain guidewire position.

Accordingly, there is a need for a microcatheter device which offsets many of these difficulties and disadvantages by having removably connectable segments where the variability of the device provides for less time and labor intensive surgical procedures by replacing multiple distinct microcatheters, increases the speed and ease of switching between techniques requiring different length microcatheters, and minimizes the need to employ specific methods to ensure the guidewire placement while changing out microcatheters.

### SUMMARY

This summary is provided to introduce in a simplified form concepts that are further described in the following detailed descriptions. This summary is not intended to identify key features or essential features of the claimed subject matter, nor is it to be construed as limiting the scope of the claimed subject matter.

An object of the present disclosure is to present a catheter device, such as a microcatheter device, that offsets various difficulties and disadvantages which currently exist and provides for quicker and easier surgical procedures through multiple removably connectable microcatheter segments. In various aspects, it is an object of the present disclosure to present a microcatheter that can replace multiple traditionally discrete microcatheters, increase the speed and ease of switching between techniques requiring microcatheters of different lengths, and minimize the need to employ specific techniques to ensure the guidewire remains in place to change out microcatheters.

In various embodiments, a catheter device comprises a distal segment, a push-wire extension, a proximal segment, a connector unit, and a motion lock hub. The distal segment also includes a body having a first end with a first opening and an opposed second end with a second opening and a hollow interior bore extending axially through the boyd between the first opening and second opening in embodiments. The push-wire extension, in embodiments, also has an attached end connected to the distal segment and an available end extending away from the attached end and distal segment. The proximal segment includes a structure having a front end with a front opening and an opposed rear end with a rear opening and a vacant interior lumen through the structure between the front opening and the rear opening, a longitudinal channel extending along a first axial portion of the structure between the front end to the rear end parallel to the lumen in embodiments. In embodiments, the longitudinal channel comprises an outlet and an opposed inlet configured to accept the available end of the push-wire extension such that a portion of the push-wire extension extends through at least a portion of the longitudinal channel such that the proximal segment slidably engages the push-wire extension. In embodiments, the device also includes a connector module configured to selectively secure the distal segment and the proximal segment together in an attached configuration when the proximal segment is slid along the push-wire extension such that the second end of the body abuts the front end of the structure. In embodiments, the interior bore and the interior lumen define a contiguous passage through the catheter device configured to accept and enclose a guidewire. The device also includes, in embodiments, a motion lock hub configured to selectively lock the push-wire extension and the proximal segment in fixed relation, where the distal segment and the proximal segment can be manipulated in unison when secured together through the connector module and when the push-wire extension and proximal segment are locked in fixed relation through the motion lock hub.

In particular embodiments, the connector module includes a housing having an aperture and an insertion member configured to be removably secured within the housing upon introduction of the insertion member through the aperture, where each of the housing and insertion member are disposed upon a discrete one of the body and the structure.

In various embodiments, the connector module includes a recess and an insertion member configured to be removably secured within the recess, where each of the recess and insertion member are defined into or disposed upon a discrete one of the body and the structure. In certain embodiments, the recess includes at least one of an orifice through, a depression in, and a channel along a surface of one of the body and the structure. In specific embodiments, the insertion member includes one of a bump, ridge, shoulder, tab, prong, and wing having a shape and size configured to cooperate with the recess.

In further embodiments, the connector module comprises a recess having a threaded surface and an insertion member having a threaded surface, where the threaded surface of the insertion member matingly engages with the threaded surface of the recess to removably secure the proximal segment to the distal segment.

In additional embodiments, the connector module is configured to cooperatively engage portions of the body and the structure. The connector module, in certain embodiments, frictionally engages portions of the body and the structure. Additionally, the connector module, in specific embodiments, includes a ring having radially protruding extensions configured to engage an aperture on each of the body and the structure. In certain embodiments, the connector module is integrally attached to one of the body and the structure to facilitate connection with the other.

In particular embodiments, the device further includes a tapered tip having a frustum shape with open parallel bases connected by a through-hole, the tapered tip disposed adjacent to the first end of the body and such that the through-hole and the interior bore are in fluid communication and wherein the contiguous passage is further defined by the through-hole. In certain embodiments, the device also includes a cylindrical marker band disposed between the body and tapered tip. In further embodiments, the tapered tip comprises a marker band portion.

In embodiments, at least a portion of an outer surface of at least one of the body, the structure, and the longitudinal channel includes a hydrophilic coating. In other embodiments, at least a portion of an inner surface of at least one of the body, the structure, and the longitudinal channel include a hydrophobic coating.

In various embodiments, the device further includes a lever device configured to removably affix to the available end of the push-wire extension for manipulation of the push-wire extension.

In particular embodiments, the push-wire extension comprises a flat or round strip having a generally square, rectangular, or round cross section.

In certain embodiments, the push-wire extension comprises a width or thickness of 0.010 or less inches.

In additional embodiments, the distal segment, the proximal segment, and the connector is configured to be inserted into and pass through an interior of a guide catheter. The distal segment, the proximal segment, and the connector module, in still further embodiments, are configured to insert into and pass through a port of a y-adapter affixed to an end of the guide catheter.

In at least one embodiment, the motion lock hub includes a handle portion affixed to a proximal end portion of a wire, where a central portion of the wire forms a periphery of a coiled wire segment and the proximal end portion is opposed to a distal end portion affixed to the structure; where the coiled wire segment is disposed within at least a portion of the longitudinal channel of the proximal segment such that the coiled wire segment is wrapped around a portion of the push-wire extension disposed within the longitudinal channel; and where the coiled wire segment is configured to tighten into frictional engagement with the portion of the push-wire extension disposed within the longitudinal channel to lock the portion of the push-wire extension and the proximal segment in fixed relation based on manipulation of a control disposed upon the handle portion. In further embodiments, the control is biased into a first position and wherein the push-wire extension within is freely slidable within the coiled wire segment and the longitudinal channel when the control is in the first position. In specific embodiments, the control is configured to be manipulated by moving the control away from the first position towards a second position wherein the coiled wire segment tightens into frictional engagement with a portion of the push-wire extension. In additional embodiments, the control is configured to be selectively secured in the second position.

In embodiments, the motion lock hub comprises a member insertable into at least one of the longitudinal channel and the interior lumen and configured to frictionally engage the structure and a portion of the push-wire extension. Indeed, the member, in certain embodiments, is wedged into the longitudinal channel to frictionally engage the push-wire extension and a portion of the structure.

In further embodiments, the longitudinal channel includes a clamp portion and the motion lock hub includes a control unit configured to adjust the clamp portion from an open position to a clamped position where the clamp portion frictionally engages the push-wire extension in the clamped position.

In additional embodiments, the motion lock hub is configured to selectively lock the push-wire extension and the proximal segment in fixed relation in conjunction with the connector module securing the distal segment to the proximal segment.

In at least one embodiment, the connector module may include a locking member disposed along a portion of the body and biased into a first secured position where the locking member engages a mating member disposed on or connected to the structure and where the push-wire extension is in operable connection with the locking member such that manipulation of the push-wire extension causes the locking member to move away from the first secured position so that the distal segment and proximal segment are separated. In particular embodiments, the locking member is biased into the first position.

In additional embodiments, the longitudinal channel is disposed adjacent an outer surface of the structure through which the interior lumen extends.

### BRIEF DESCRIPTION OF THE DRAWINGS

The foregoing, as well as the following Detailed Description, is better understood when read in conjunction with the appended Figures. For the purposes of illustration, there are shown in the figures certain exemplary embodiments; however, the presently disclosed subject matter is not limited to the specific methods and instrumentalities disclosed.

The embodiments illustrated, described, and discussed herein are illustrative of the present invention. As these embodiments of the present invention are described with reference to illustrations, various modifications or adaptations of the methods and or specific structures described may become apparent to those skilled in the art. It will be appreciated that modifications and variations are covered by the above teachings and within the scope of the appended claims without departing from the spirit and intended scope thereof. All such modifications, adaptations, or variations that rely upon the teachings of the present invention, and through which these teachings have advanced the art, are considered to be within the spirit and scope of the present invention. Hence, these descriptions and drawings should not be considered in a limiting sense, as it is understood that the present invention is in no way limited to only the embodiments illustrated.

Embodiments of the present invention are shown with reference to the following drawings introduced as follows:

FIG. 1 is a side elevation view of portions of a multi-segmented microcatheter device according to one or more embodiments including a distal segment having a tapered tip, marker band, push-wire extension with affixed lever device, and a housing of a connector module;

FIG. 2A is a side elevation view of a lever device connected to the available end of a push-wire extension of a multi-segmented microcatheter device according to one of more embodiments;

FIG. 2B is a side elevation view of a lever device unconnected from the available end of a push-wire extension of a multi-segmented microcatheter device according to one or more embodiments;

FIG. 3 is a side elevation view of a portion of a multi-segmented microcatheter device according to one of more embodiments showing a distal segment having a housing and push-wire extension and a proximal segment having a longitudinal channel slidably engaged to the push-wire extension, an insertion member ready to engage the housing, and a portion of the guidewire extending through the respective interior bore and lumen of each;

FIG. 4 is a second end elevation view of the distal segment of a multi-segmented microcatheter device according to one of more embodiments showing the push-wire extension affixed to a bottom portion of the hollow tubular body, a housing affixed to a top portion of the body, and the interior bore through the distal segment;

FIG. 5 is a front end elevation view of the proximal segment of a multi-segmented microcatheter device according to one or more embodiments showing an inlet to the longitudinal channel for the push-wire extension along a bottom portion of the proximal segment and an insertion member along a top portion of the proximal segment to mate with a housing on the distal segment, and an interior lumen through the proximal segment;

FIG. 6 is a top plan view of the proximal segment of a multi-segmented microcatheter device according to one or more embodiments showing a motion lock hub member that connects to both the push-wire extension and the proximal segment;

FIG. 7A is a cross sectional view of a coiled wire segment of a multi-segmented microcatheter device according to one or more embodiments showing the push-wire extension therethrough;

FIG. 7B is an elevation view of a distal segment and proximal segment of a multi-segmented microcatheter device according to one or more embodiments joined together and having a motion lock hub including a handle portion affixed to a wire forming a coiled segment through which the push-wire extension passes wherein rotation of the handle portion causes frictional engagement of the push-wire extension to lock the proximal segment and push-wire extension together;

FIG. 8 is a diagram of a step in an angioplasty prior to utilizing a multi-segmented microcatheter device according to one or more embodiments wherein a guidewire is positioned through a sheath, or guide catheter, to a position adjacent a coronary lesion;

FIG. 9 is a diagram of a step in an angioplasty utilizing a multi-segmented microcatheter device according to one or more embodiments wherein the microcatheter device having the distal and proximal segments connected together is passed along the guidewire through the sheath, or guide catheter, towards the coronary lesion;

FIG. 10 is a diagram of a step in an angioplasty utilizing a multi-segmented microcatheter device according to one or more embodiments wherein the microcatheter device and guidewire of FIG. 9 is advanced through the coronary lesion, with the microcatheter providing support;

FIG. 11 is a diagram of a step in an angioplasty utilizing a multi-segmented microcatheter device according to one or more embodiments wherein the proximal segment has been unconnected from the distal segment and removed, leaving the distal segment traversing the coronary lesion;

FIG. 12 is a diagram of a step in an angioplasty utilizing a multi-segmented microcatheter device according to one or more embodiments wherein the distal segment has been removed without requiring the guidewire to be trapped to ensure it remains through the coronary lesion;

FIG. 13 is a side elevation view of a ring connector module of a multi-segmented microcatheter device according to one or more embodiments having protruding extensions configured to engage apertures in the proximal and distal segments to secure them together; and

FIG. 14 is a side elevation view of a ring connector module of a multi-segmented microcatheter device according to one or more embodiments where the protruding extensions are engaging apertures in the proximal and distal segments to secure them together.

### DETAILED DESCRIPTION

The following description and figures are illustrative and are not to be construed as limiting. Numerous specific details are described to provide a thorough understanding of the disclosure. In certain instances, however, well-known, or conventional details are not described to avoid obscuring the description. Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that same thing can be said in more than one way.

Alternative language and synonyms may be used for any one or more of the terms discussed herein. No special significance is to be placed upon whether a term is elaborated on or discussed herein. Synonyms for certain terms are provided. A recital of one or more synonyms does not exclude the use of other synonyms. The use of examples anywhere in this specification, including examples of any terms discussed herein, is illustrative only, and is not intended to further limit the scope and meaning of the disclosure or of any exemplified term. Likewise, the disclosure is not limited to various embodiments given in this specification.

Without intent to limit the scope of the disclosure, examples of instruments, apparatus, methods, and their related results according to the embodiments of the present disclosure are given below. Note that titles or subtitles may be used in the examples for convenience of a reader, which in no way should limit the scope of the disclosure.

### Overview

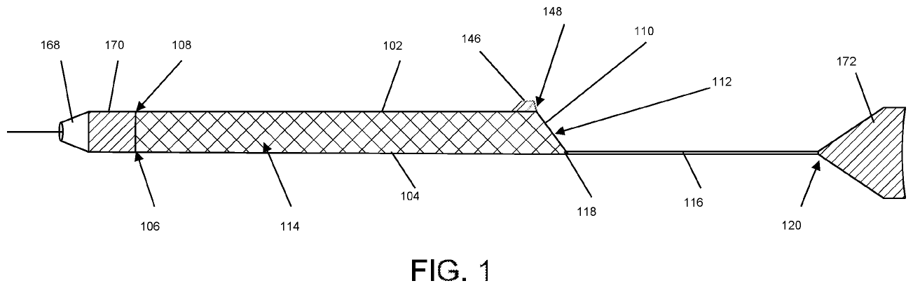

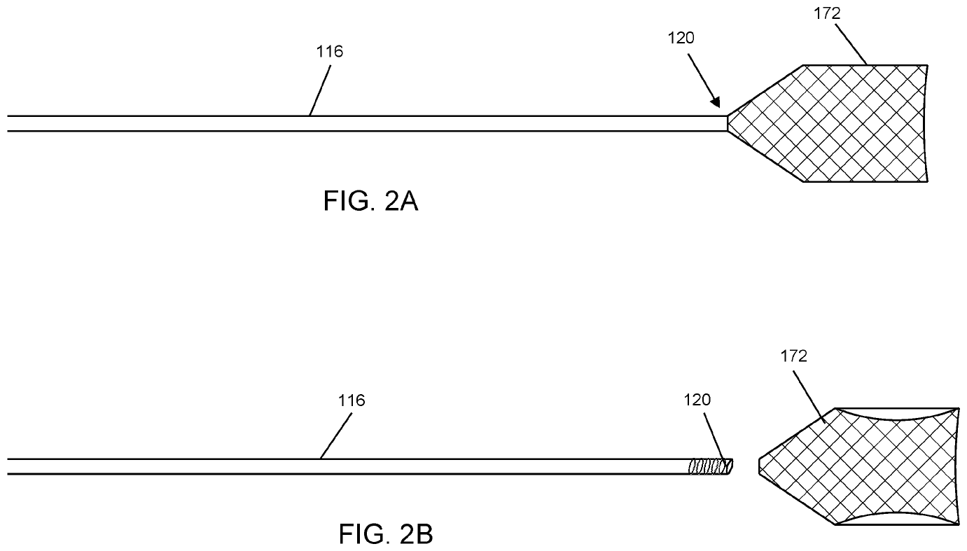

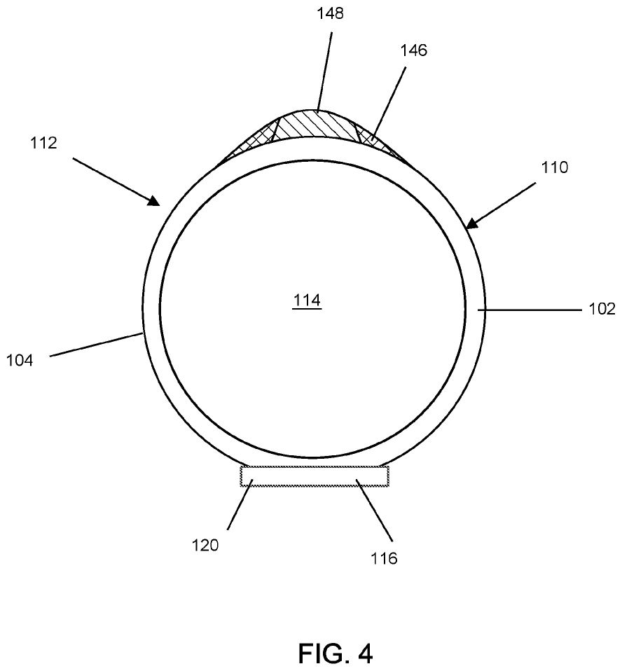

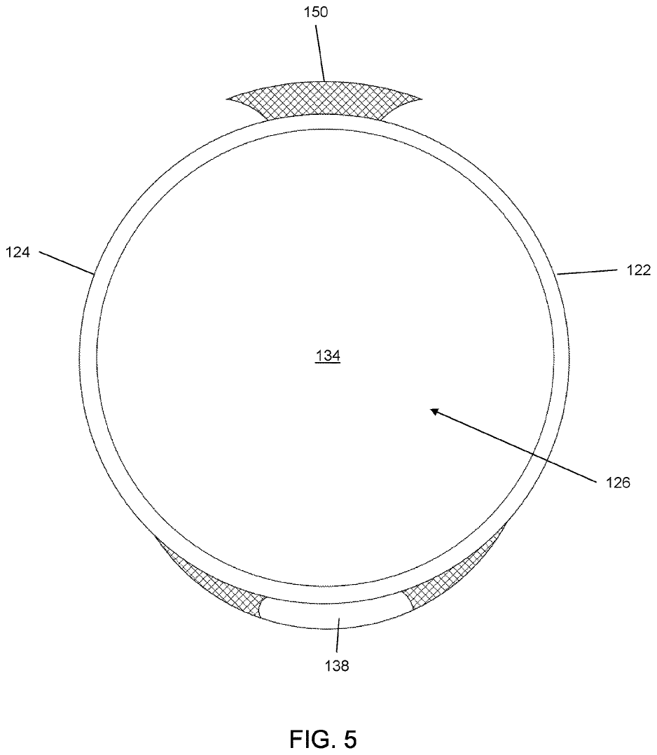

As shown in FIGS. 1-7B, embodiments of a catheter device 100, such as a multi-segmented microcatheter device, include multiple hollow segments 102, 122 disposed in series to define a contiguous passage 136 through the hollow segments 102, 122. The device, in embodiments, further has a push-wire extension 116 protruding away from the lead distal segment 102 and a longitudinal channel 138 along each of the following proximal segments 122 that slidably engages the push-wire extension 116 to be guided into position relative to the other segment(s). Generally, the device 100 also has a connector module 144 configured to hold two abutting segments together and a motion lock hub 156 configured to connect and secure the push-wire extension 116 and at least one of the following proximal segments 122 having a longitudinal channel 138 thereupon.

Specifically, with respect to FIGS. 1-7B, embodiments of the device 100 include a distal segment 102 having a body 104 with a first end 106 with a first opening 1108, second end 110 with a second opening 112, and a hollow interior bore 114 between, a push-wire extension 116 protruding longitudinally away from the second end 106 of the distal segment 102. Moreover, embodiments of the device 100 also include a proximal structure 124 with a front end 126 having a front opening 128, rear end 130 having a rear opening 132, and a vacant interior lumen 134 therebetween, and a longitudinal channel 138 sized and shaped to accept and slidably engage a portion of the push-wire extension 116 running along a first axial portion of the structure 124 between the front end 126 to the rear end 130 and parallel to the interior lumen 134. Further, embodiments of the device 100 also include a connector module 144 configured to removably and selectively secure a portion the distal segment 102 and the proximal segment 122 in an attached configuration and a motion lock hub 156 configured to selectively lock the push-wire extension 116 and the proximal segment 122 in fixed relation. In embodiments, the interior bore 114 and interior lumen 134 form a contiguous passage 136 through which one or more specific types of the guidewires 176 may extend and the distal segment 102 and the proximal segment 122 can be manipulated in unison when secured together through the connector module 144 and when the push-wire extension 116 and proximal segment 122 are locked in fixed relation through the motion lock hub 15+.

### Elements

While various features and elements have been described in reference to particular embodiments and variations above, it is to be understood that no limitation of the scope of this disclosure is hereby intended. Thereby, elements and features might be utilized in any combination and for any embodiment to which it is particularly useful. To further promote understanding of the principles of the present disclosure, additional discussion related to particular elements of the present discussion is provided below.

### Distal Segment

As shown in FIGS. 1, 3, 4, and 7B, the device 100 has a lead distal segment 102 with a first end 106 having a first opening 108, a second end 110 having a second opening 112, and a hollow interior bore 114 therebetween in embodiments. The distal segment 102 comprises a microcatheter portion that can be utilized independently or in conjunction with one or more additional following proximal segments 122 removably affixed thereto in series and so a contiguous passage 136 is formed from their hollow interior portions in embodiments.

As shown, the distal segment 102 typically has a hollow tubular body 104. However, it is understood that the distal segment 102 may be any shape useful for a particular procedure or with particular devices, such as sheaths, guide catheters 174, and guidewires 176. Indeed, the exterior surface of the body 104 may be fluted or may have a rectangular cross-section, in embodiments. In certain embodiments, the cross-sectional shape of the exterior surface of the distal segment 102 may be different for axial different axial portions.

In embodiments, the distal segment 102 includes a coating along at least a portion of one or more of the surfaces thereof. For example, in FIG. 1 the distal segment 102 comprises a hydrophilic coating on an outer surface thereof. Additionally, the distal segment 102 may have a coating on a portion of an inner surface thereof. For example, in at least one embodiment a separate coating is disposed on an inner and outer surface of a segment. Moreover, while a hydrophilic coating has been discussed herein, it is foreseen that one or more portions, such as surfaces, of the distal segment may have a hydrophobic or other type of coating as may be desired and may enhance functionality of the device. For example, in an embodiment the outer surface of the distal segment 112 comprises a hydrophilic coating and an inner surface thereof comprises a hydrophobic coating.

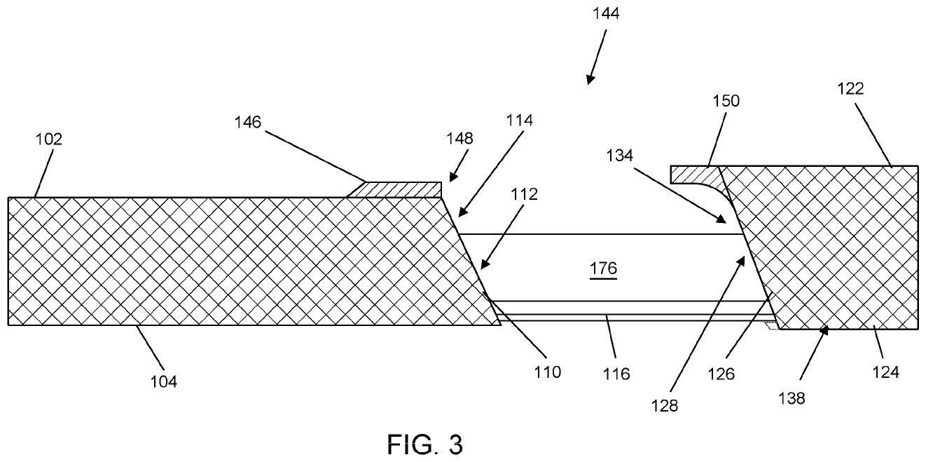

In the embodiments of FIGS. 1 and 3, the second end 110 of the distal segment 102 may have an angled edge, textured edge, or uneven edge, which mates with a matching edge of an abutting proximal segment 122, to facilitate the alignment of segments together, along with any mating sections of the connector module 144.

### Tapered Tip

As shown in FIG. 1, the device 100 may have a tapered tip 168 disposed at the first end 106 of the distal segment 102, opposite to the second end 110 from which the push-wire extension 116 protrudes in embodiments. In embodiments, the tapered tip 168 may be removably affixed to the distal segment 102, allowing for variation of the tip 168 to be utilized and allowing for the device to be utilizable with a variety of sized guidewires 176.

In embodiments, the tip 168 is hollow and conical or has at least a portion with a frustum shape with open parallel bases, allowing for a guidewire 176 to pass therethrough. However, it is understood that the tip 168 may be any shape that is useful for a particular procedure or with particular devices. For example, the tip 168 may have a portion that has a different tip 168 entry profile and/or different tapers which may be more abrupt or gradual along a longitudinal axis. Moreover, it is foreseen that at least one of the openings of the tapered tip 168 may be sized and shaped to align with a particular gauge, or size, guidewire 176 in embodiments. For example, the tip 168 may have an opening sized slightly larger than the guidewire wherein the tip 168 helps the distal segment 102 provide support for a guidewire 176 during traversal through a lesion or blockage.

### Marker Band

As shown in FIG. 1, the device 100 may have a marker band 170 disposed between the first end 106 of the distal segment 102 and the conical tip 168 in embodiments. The marker band 170 is, in embodiments, made from a radiopaque material that allows for the tip 168 to be identified in imaging. In certain embodiments, the marker band 170 may be an integrally formed part of either the tapered tip 168 or the body 104. However, in other embodiments, the marker band 170 may be a separable portion that is affixed to both the first end 106 of the body 104 on one side and the tapered tip 168 on another. In at least one embodiment, the tapered tip 168 may be made of or with the same material as the marker band 170, and the marker band 170 itself may be omitted from the device 100.

### Push-Wire Extension

As shown in FIGS. 1-4, and 7B, embodiments of the device 100 have a push-wire extension extending away from the second end 110 of the distal segment 102 which provides the ability to manipulate the distal segment 102, and the device 100 in aspects. As shown in FIG. 3, the push-wire extension 116 also, in embodiments, provides a guide for additional proximal segments 122 to be positioned relative to the leading distal segment 102. Indeed, in embodiments, the push-wire extension 116 is slidably engaged by a longitudinal channel 138 disposed along at least one following proximal segments 122. Thereby, the proximal segment 122 may be guided along the push-wire extension 116 into place behind the distal segment 102 such that the connector module 144 is aligned to removably secure the distal segment 102 with the proximal segment 122. In embodiments, at least a portion of the push-wire extension 116 protrudes away from the distal segment 102 along a line parallel to the interior bore 114 and, in embodiments, along a longitudinal axis that passes through the first end 106, and second end 110, as shown in FIG. 1.

In embodiments, the push-wire extension 116 comprises a flat strip having a square or rectangular or round cross section and a width or thickness of 0.010 inches or less. However, it is understood that the push-wire extension 116 may be any shape or size useful for a particular procedure or with particular devices.

Moreover, in embodiments, the push-wire extension 116 may have a lever device 172 configured to removably affix to an available end 120, opposite the attached end 118 affixed to the distal segment 102, to aid a user in manipulating the push-wire extension 116 and the distal segment 102 or whole device 100. In embodiments, the lever device 172 may have a recess with a threaded surface to mate with a threaded surface portion adjacent the available end 120. In alternative embodiments, the lever device 172 has a selectively engageable latch which is secured to bumps, ridge, protrusions, striations, or some other feature along the surface of the push-wire extension 116 adjacent the available end 120. Alternatively, the lever device 172 may just clamp to a portion of the push-wire extension 116.

In at least one embodiment, the push-wire extension 116 forms part of a control for a connector module 144, discussed below. That is, in embodiments, the connector module 144 may selectively engage and disengage based on a user manipulating the push-wire extension 116. For example, the attached end 118 of the push-wire extension 116 may be affixed to a movable tooth portion formed in the wall of the distal segment 102 such that the tooth is biased into a connection position to engage a respective aperture 148 or mating portion, such as an insertion member 150, of the proximal segment 122 but will unlock if the push-wire extension 116 is manipulated by the user.

### Proximal Segment

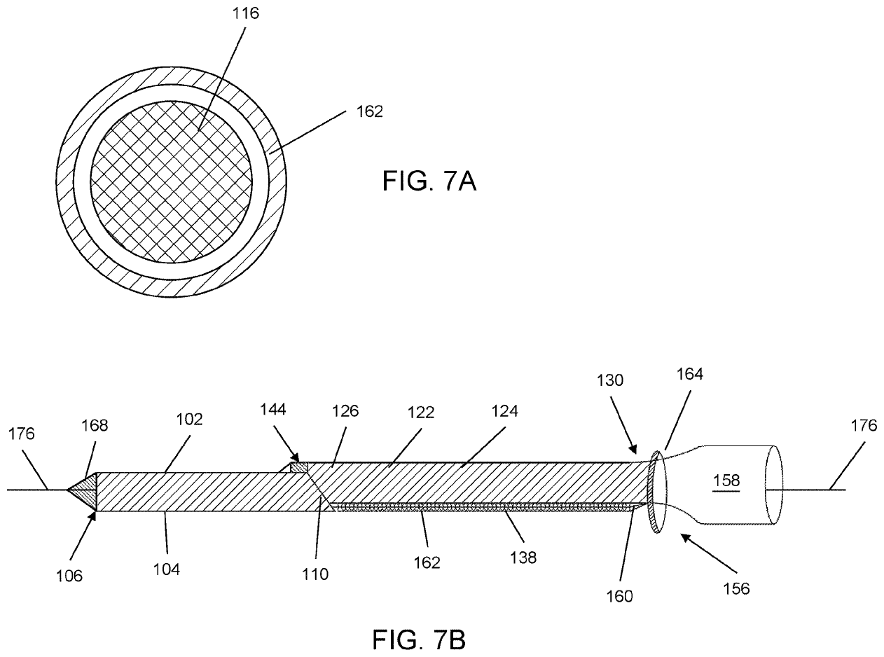

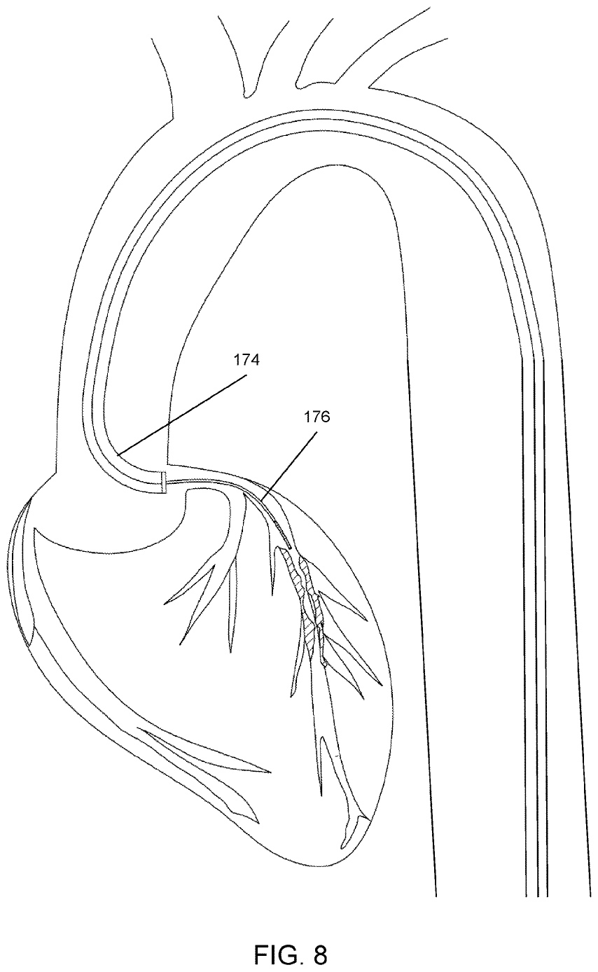

As shown in FIGS. 3 and 7B, the device 100 has a following proximal segment 122 with a structure 124 including a front end 126, a rear end 130, and a vacant interior lumen 134 therebetween, in embodiments. The proximal segment 122 comprises a microcatheter portion that is utilized in conjunction with a distal segment 102. In particular, the proximal segment 122 is selectively and removably affixed to the distal segment 102 in series and so a contiguous passage 136 is formed from the interior bore 114 of the distal segment 102 and the interior lumen 134 of the proximal segment 122.

As indicated in FIGS. 3, 5, 7A and 7B, the proximal segment 122 includes a longitudinal channel 138 having a shape and size to enclose and slidably engage the push-wire extension 116 extending along a first axial portion of the structure 124 between the front end 126 and the rear end 128 and parallel to the interior lumen 134. Thereby, the proximal segment 122 may be guided into position to abut the distal segment 102 so that the connector module 144 is appropriately aligned by the push-wire extension 116. In embodiments, the longitudinal channel 138 is discrete from the interior lumen 134 such that the lumen 134 and the longitudinal channel 138 are not in fluid communication. In embodiments, the longitudinal channel 138 and interior lumen 134 may be in fluid communication but still configured to keep the push-wire extension 116 separate from the guide wire 176 or the interior lumen 134. In certain embodiments, the longitudinal channel 138 may be defined within the structure 124, such as being disposed along a portion of the inner surface of the structure 124 of the proximal segment 122 which defines the interior lumen 134. In other embodiments, the longitudinal channel 138 may be defined outside the structure 124, such as being disposed along a portion of the outer surface of the structure 124 of the proximal segment 122.

As shown, the proximal segment 122 typically has a hollow cylindrical structure 124. However, it is understood that the proximal segment 122 may be any shape that is useful for a particular procedure or with particular devices, such as sheaths, guide catheters 174, and guidewires 176. Indeed, the exterior surface of the structure 124 may be fluted or may have a rectangular cross-section, in embodiments. In certain embodiments, the cross-sectional shape of the exterior surface of the proximal segment 122 may be different for axial different axial portions.

In embodiments, the proximal segment 122 includes a coating along at least a portion of one or more of the surfaces thereof. For example, just as for the distal segment 102 in FIG. 1, the proximal segment 122 may comprise a hydrophilic coating on an outer surface thereof. Additionally, the proximal segment 122 may have a coating on a portion of an inner surface thereof. For example, in at least one embodiment a separate coating is disposed on an inner and outer surface of a segment. Moreover, while a hydrophilic coating has been discussed herein, it is foreseen that one or more portions, such as surfaces, of the proximal segment 122 may have a hydrophobic, hydrophilic, or other type of coating as may be desired and may enhance functionality of the device 100. For example, in an embodiment the outer surface of the proximal segment 122 comprises a hydrophilic coating and an inner surface thereof comprises a hydrophobic coating.

In embodiments like that of FIG. 3, the front end 126 of the proximal segment 122 may have an angled edge, textured edge, or uneven edge, which mates with a matching edge of the distal segment 102 to which it abuts, to facilitate the alignment of segments together, along with any mating sections of the connector module 144, along with the longitudinal channel 138.

In embodiments, the longitudinal channel 138 may have a clamp portion that is capable of frictionally engaging the push-wire extension 116. In such embodiments, the motion lock hub 156 may have a control unit connected in operable communication with the clamp portion to selectively cause such portion of the longitudinal channel 138 to frictionally engage the push-wire extension 116. That is, certain portions of the motion lock hub 156 may be disposed within, around, or adjacent to the longitudinal channel 138 to facilitate the connection of the such portions with the push-wire extension 116.

### Connector Module

As shown in FIGS. 1, 3-5, and 7B, the device 100 further includes a connector module 144 configured to removably connect two segments, such as the distal segment 102 and proximal segment 122. In embodiments, such as those of FIGS. 1, 3-5, and 7, the connector module 144 may comprise a housing 146 having an aperture 148 disposed along the body 104 at or near the second end 110 thereof and an insertion member 150 extending away from the front end 126 of the structure 124 and configured to selectively mate and engage with the housing 146. In additional embodiments, the connector module 144, such as the housing 146 thereof, may have an aperture 148 along a portion of the distal segment 102 or proximal segment 122 and an insertion member 150 which inserts into and mates with the aperture 148. In embodiments, the aperture 148 may be an opening through, a depression in, and a channel along a surface of one of the segments. In additional embodiments, the insertion member 150 may comprise one of a bump, ridge, shoulder, tab, prong, and wing having a shape and size configured to cooperate with the aperture 148. For example, you could have tabs which are attached to a proximal segment 122 and mate with holes along a distal segment 102. In further embodiments, the connector module 144 may comprise two mating threaded surfaces, one along each segment.

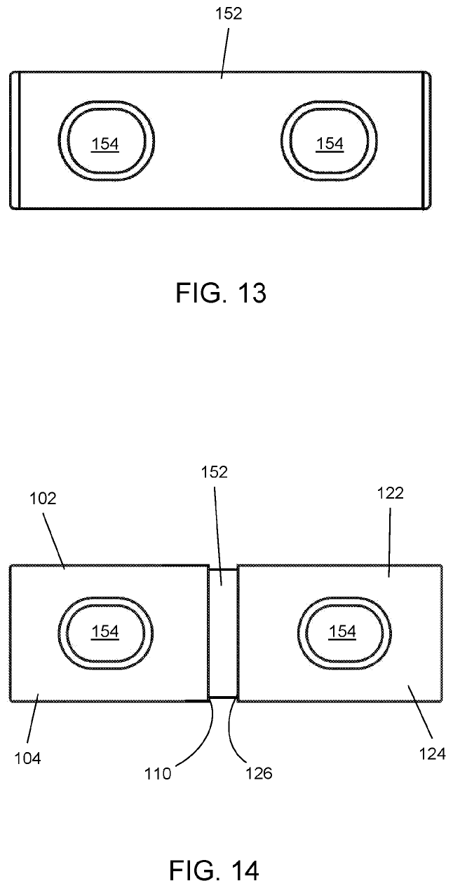

Moreover, in additional embodiments, the connector module 144 may be an independent unitary member, as in FIGS. 13 and 14, which has a portion to engage the distal segment 102 and a portion to engage the proximal segment 122. In specific embodiments, the engaging portions of the connector module 144 frictionally engage each respective segment. Moreover, the connector module 144 can comprise a ring 152 having radially protruding extensions 154 configured to engage apertures 148 on or along each of the surfaces of the distal segment 102 and proximal segment 122.

Accordingly, in embodiments, the connector module 144 may be an independent separable member from both segments 102, 122 or one or more portions may be part of or integrally formed with one or more of the segments 102, 122. In embodiments, the connector module 144 may utilize a number of methods to connect with one or more segments 102, 122. Indeed, in embodiments the connector module 144 may be a separate member which frictionally engages each of the distal segment 102 and proximal segment 122.

### Motion Lock Hub

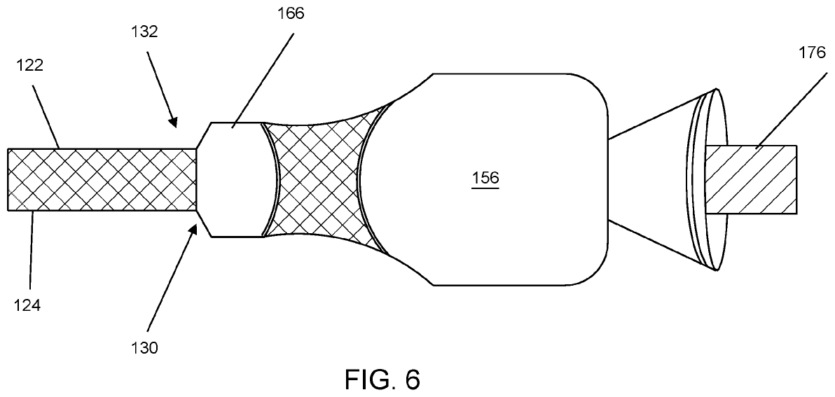

As shown in FIGS. 6, 7A and 7B, the device 100 further includes a motion lock hub 156 configured to selectively lock the push-wire extension 116 and the proximal segment 122 in fixed relation. In embodiments, utilization of the motion lock hub 156 to lock the push-wire extension 116 and the proximal segment 122, along with the connector module 144, allows the distal segment 102and proximal segment 122 to be manipulated in unison. The motion lock hub 156, in embodiments, comprises a member 166 that affixes to both the proximal segment 122, about a rear end 130 thereof, and the push-wire extension 116.

In at least one embodiment, the motion lock hub 156 may comprise a handle portion 158 that affixes to a wire 160 end, the wire 160 having a central portion that forms a coiled wire segment 162 and another end that affixes to the proximal segment 122. In embodiments, the coiled wire segment 162 may form part of, or be disposed within, the longitudinal channel 138 of the proximal segment 122, so that the push-wire extension 116 passes through a central portion, or void, of the coiled wire segment 162, as in FIG. 7A. Thereby, in embodiments, the coiled wire segment 162 may move from a loosened position to an engaged position, where the coiled wire segment 162 frictionally engages the push-wire extension 116, when the handle portion 158, or a control 164 thereupon, is manipulated, as in FIG. 7B. In embodiments, the handle portion 158 itself may have a control 164 thereupon which, when manipulated, moves the coiled wire segment 162 between the loosened position and the engaged position. In embodiments, the control 164 may be manipulated by pulling on it or turning it. The handle portion 158, or control 164, may also, be locked into an engaged position in embodiments. To aid operation, the handle portion 158 or control 164 may be biased into the loosened position, such as by the natural form of the coiled wire segment 162.

In further embodiments, the motion lock hub 156 may be a separate member 166 that is inserted into the longitudinal channel 138 to hold the push-wire extension 116 in place, such as a wedge. The separate member 166, in embodiments, may be shaped and formed to engage a portion, such as the interior, of the proximal segment 122 as well. In additional embodiments, the motion lock hub 156 may be a separate member 166 that clamps or otherwise holds the push-wire extension 116 while engaging the proximal segment 122.

Moreover, the motion lock hub 156 may further comprise a control unit which causes a portion of the longitudinal channel 138, such as a clamp portion, to move from an open position to a clamped position which frictionally engages the push-wire extension 116.

In embodiments, the motion lock hub 156 may be operably connected with the connector module 144 such that engagement of the connector module 144 to attach the distal segment 102 and proximal segment 122 causes the motion lock hub 156, in conjunction, to lock the proximal segment 122 and push-wire extension 116 into to a fixed relation.

Similarly to the connector module 144, the motion lock hub 156 may utilize a number of methods to connect the proximal segment 122 and push-wire extension 116 in embodiments.

### Embodiments of Use

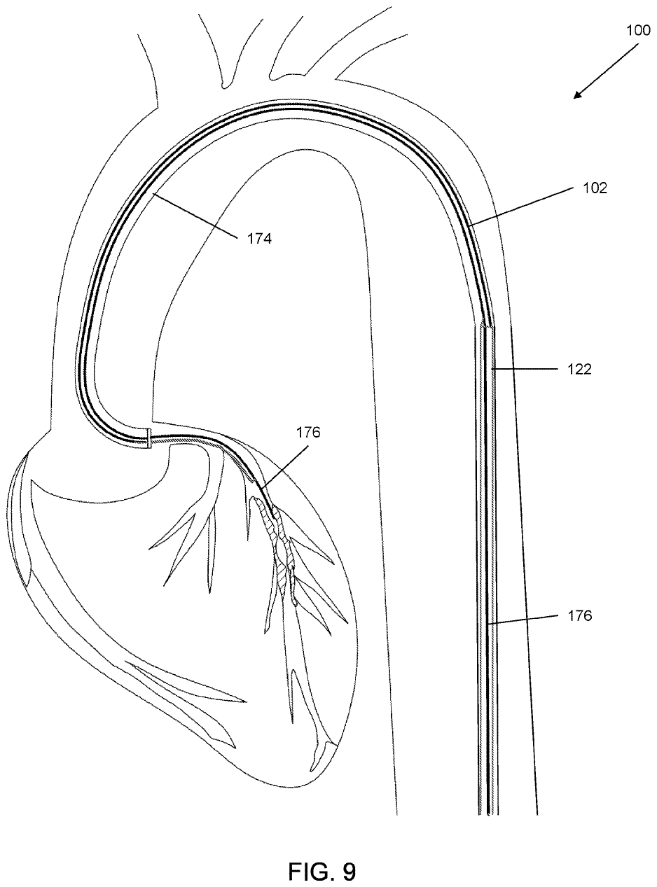

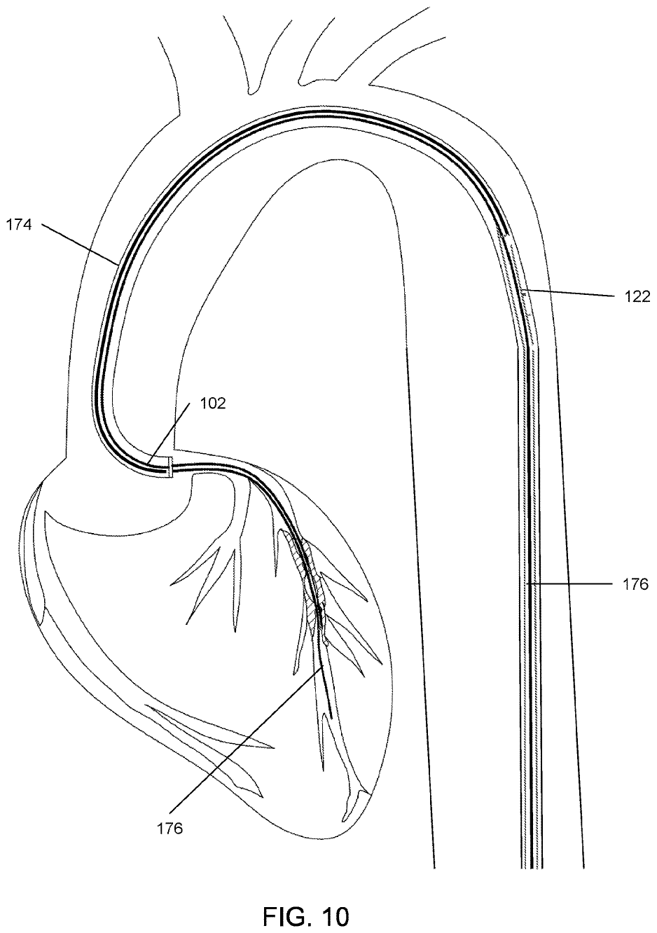

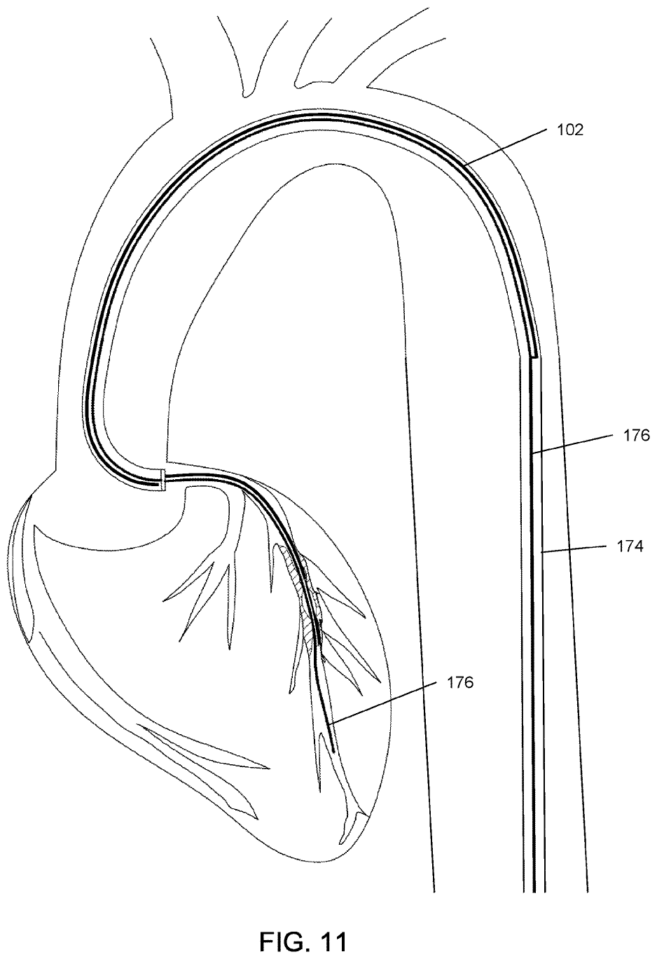

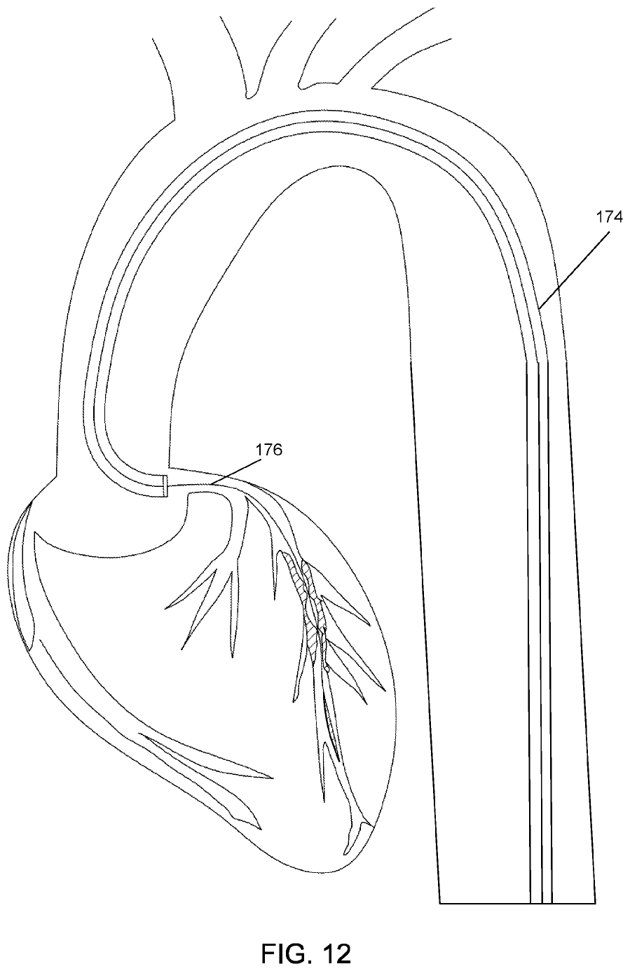

One or more embodiments of use of a multi-segmented microcatheter device 100 are shown in the steps of FIGS. 8-12. Indeed, FIG. 8 shows a situation where a guide catheter 174, or sheath, has been inserted into the circulatory system such that an end thereof is disposed near the coronary tree and a guide wire has been inserted into the arterial segment having a coronary lesion which it is unable to pass. To provide support for the guidewire 176 so it can pass the lesion, a user can load a microcatheter device 100 having a connected proximal segment 122 and distal segment 102, and an activated motion lock hub 156, onto the guidewire 176 and direct the device 100 into position to support the guidewire 176 for movement through the lesion, as in FIG. 9. That is, the device 100, having connected segments 102, 122 and a secured motion lock hub 156, is pushed through the Y-adapter portion (not shown) of the guide catheter 174 and down through the guide catheter 174 into position. Once positioned, the device 100 can be advanced, pushed or torqued, into the vasculature, e.g., through the lesion, as shown in FIG. 10. The microcatheter device 100 provides support to aid the guidewire 176 in crossing the lesion and, once crossed, may provide a channel through which the guidewire 176 may be swapped out for another guidewire 176, such as a workhorse guidewire 176. Otherwise, the motion lock hub 156 may be unsecured and the proximal segment 122and distal segment 102 may be separated to allow the proximal segment 122 to be removed over the push-wire extension 116 until it is backed out through the guide catheter 174 and the Y-adapter portion (not shown), as in FIG. 11. Thereafter, the distal portion 102 may be removed and guidewire 176 may be left in place, without requiring any trapping techniques to hold the guidewire 176, as in FIG. 12. At such portion, the guidewire 176 is in place and ready for interventional devices, such as balloons or stents, to be delivered.

In another embodiment of use, only the distal segment 102 of the microcatheter device 100 may be loaded onto and guided down the guidewire 176 in the situation of FIG. 8. That is, the distal segment 102 is passed through the Y-adapter portion of the guide catheter 174 and through the guide catheter 174 along the guidewire 176 until it is in place, similar to FIG. 11, except that the guidewire 176 has not yet crossed the lesion. In such an instance, the distal portion 102 may provide sufficient support to allow the guidewire 176 to cross the lesion, without an attached proximal segment 122. However, if an operator is unable to cross the lesion and decides to also connect the proximal segment 122, providing additional support to the guidewire 176 for advancement through the lesion, the proximal segment 122 may be loaded onto the guidewire 176 and the available end of the push-wire extension 116 passed through the longitudinal channel 138, and the proximal segment 122 may be slid through the Y-adapter portion and other portion of the guide catheter 174 until it abuts the distal segment 102. Upon proper alignment, the connector module 144 may attach the proximal segment 122 and distal segment 102 and the motion lock hub 156 may engage. Thereafter, the microcatheter device 100 may proceed to be utilized in the way demonstrated by FIG. 10 above and, further on, broken down and removed as described with respect to FIGS. 11 and 12. Thereby, the disclosed multi-segmented microcatheter device 100 may be quickly and easily adjusted to operate with both over-the-wire and rapid exchange techniques.

While various features and elements have been described in general above, it is to be understood that no limitation of the scope of this disclosure is hereby intended. Thereby, elements and features might be utilized in any combination and for any embodiment to which it is particularly useful.

Any dimensions expressed or implied in the drawings and these descriptions are provided for exemplary purposes. Thus, not all embodiments within the scope of the drawings and these descriptions are made according to such exemplary dimensions. The drawings are not necessarily made to scale. Thus, not all embodiments within the scope of the drawings and these descriptions are made according to the apparent scale of the drawings regarding relative dimensions in the drawings. However, for each drawing, at least one embodiment is made according to the apparent relative scale of the drawing.

The descriptions of the various embodiments of the present disclosure have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first clement, without departing from the scope of the present inventive subject matter. As used herein, the term “and/or” includes all combinations of one or more of the associated listed items.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

It will be understood that when an element or layer is referred to as being “on” another element or layer, the element or layer can be directly on another element or layer, or intervening elements or layers may also be present. In contrast, when an element is referred to as being “directly on” another element or layer, there are no intervening elements or layers present. As used herein, the term “and/or” includes all combinations of one or more of the associated listed items.

Spatially relative terms, such as “below,” “beneath,” “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation, in addition to the orientation depicted in the figures. Throughout the specification, like reference numerals in the drawings denote like elements.

Embodiments of the inventive subject matter are described herein with reference to plan and perspective illustrations that are schematic illustrations of idealized embodiments of the inventive subject matter. As such, variations from the shapes of the illustrations as a result, for example, of manufacturing techniques and/or tolerances, are to be expected. Thus, the inventive subject matter should not be construed as limited to the shapes of objects illustrated herein, but should include deviations in shapes that result, for example, from manufacturing. Thus, the objects illustrated in the figures are schematic in nature and their shapes are not intended to illustrate the actual shape of a region of a device and are not intended to limit the scope of the inventive subject matter.

The terminology used herein is for the purpose of describing embodiments only and is not intended to be limiting of the present inventive subject matter. As used herein, the singular forms “a,” “an” and “the” are intended to include the plural forms as well, unless the context clearly indicates otherwise. It will be further understood that the terms “comprises” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this present inventive subject matter belongs. It will be further understood that terms used herein should be interpreted as having a meaning that is consistent with their meaning in the context of this specification and the relevant art and will not be interpreted in an idealized or overly formal sense unless expressly so defined herein. The term “plurality” is used herein to refer to two or more of the referenced items. Although any methods, devices, and materials similar or equivalent to those described herein can be used in the practice or testing of the presently disclosed subject matter, representative methods, devices, and materials are now described.

In the drawings and specification, there have been disclosed typical preferred embodiments of the inventive subject matter and, although specific terms are employed, they are used in a generic and descriptive sense only and not for purposes of limitation, the scope of the inventive subject matter being set forth in the following claims.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description but is not intended to be exhaustive or limited to the forms herein disclosed. Many modifications and variations will be apparent to those of ordinary skill in the art without departing from the scope and spirit of the disclosure. The embodiment was chosen and described to best explain the principles of the disclosure and the practical application, and to enable others of ordinary skill in the art to understand the disclosure for various embodiments with various modifications as are suited to the particular use contemplated.

1. A catheter device comprising:
a distal segment comprising a body having
a first end having a first opening,
an opposed second end having a second opening, and
an interior bore,
wherein the interior bore extends axially through the body between the first opening and the second opening;
a push-wire extension having
an attached end affixed to a portion of the distal segment and
an available end extending away from the attached end and the distal segment;
a proximal segment comprising a structure having
a front end having a front opening,
an opposed rear end having a rear opening, and
an interior lumen, wherein the interior lumen extends axially through the structure between the front opening and the rear opening, and
a longitudinal channel extending along a first axial portion of the structure between the front end and the rear end thereof and parallel to the lumen,
wherein the longitudinal channel comprises an outlet and opposed inlet configured to accept the available end of the push-wire extension such that a portion of the push-wire extension extends through at least a partial length of the longitudinal channel such that the proximal segment slidably engages the push-wire extension;
a connector module configured to selectively secure the distal segment and the proximal segment together in an attached configuration in response to the proximal segment being slid along the push-wire extension such that the front end of the structure abuts the second end of the body,
wherein the interior bore and the interior lumen define a contiguous passage through the catheter device in the attached configuration and wherein the contiguous passage is configured to accept and enclose a guidewire; and
a motion lock hub configured to selectively lock the push-wire extension and the proximal segment in fixed relation,
whereby the distal segment and the proximal segment are configured to be manipulated in unison when secured in the attached configuration with the connector module and when the push-wire extension and the proximal segment are locked in fixed relation with the motion lock hub.

2. The catheter device of claim 1, wherein the connector module comprises a housing having an aperture and an insertion member configured to be removably secured within the housing upon introduction of the insertion member through the aperture, wherein each of the housing and the insertion member are disposed upon a discrete one of the body and the structure.

3. The catheter device of claim 1, wherein the connector module comprises an aperture and an insertion member configured to be removably secured within the aperture, wherein each of the aperture and the insertion member are defined into or disposed upon a discrete one of the body and the structure.

4. The catheter device of claim 3, wherein the aperture comprises at least one of an orifice through, a depression in, and a channel along a surface of one of the body and the structure.

5. The catheter device of claim 3, wherein the insertion member comprises one of a bump, ridge, shoulder, tab, prong, and wing having a shape and size configured to cooperate with the aperture.

6. The catheter device of claim 1 wherein the connector module comprises an aperture having a threaded inner surface and an insertion member having a threaded outer surface, wherein the threaded outer surface of the insertion member matingly engages with the threaded inner surface of the aperture to removably secure the proximal segment to the distal segment.

7. The catheter device of claim 1, wherein the connector module is configured to cooperatively engage portions of the body and the structure.

8. The catheter device of claim 7, wherein the connector module frictionally engages portions of the body and the structure.

9. The catheter device of claim 7, wherein the connector module comprises a ring having opposed radially protruding extensions configured to engage an aperture defined in each of the body and the structure.

10. The catheter device of claim 7, wherein the connector module is integrally attached to one of the body and the structure.

11. The catheter device of claim 1, wherein the motion lock hub comprises:
a handle portion affixed to a proximal end portion of a wire, wherein a central portion of the wire forms a periphery of a coiled wire segment and the proximal end portion is opposed to a distal end portion affixed to the structure;
wherein the coiled wire segment is disposed within at least a portion of the longitudinal channel of the proximal segment such that the coiled wire segment is wrapped around a portion of the push-wire extension disposed within the longitudinal channel; and
wherein the coiled wire segment is configured to tighten into frictional engagement with the portion of the push-wire extension disposed within the longitudinal channel to lock the portion of the push-wire extension and the proximal segment in fixed relation based on manipulation of a control disposed upon the handle portion.

12. The catheter device of claim 11, wherein the control is biased into a first position and wherein the portion of the push-wire extension is freely slidable within the coiled wire segment and the longitudinal channel when the control is in the first position.

13. The catheter device of claim 12, wherein the control is configured to be manipulated by moving the control away the first position towards a second position wherein the coiled wire segment tightens into frictional engagement with the portion of the push-wire extension.

14. The catheter device of claim 13, wherein the control is configured to be selectively secured in the second position.

15. The catheter device of claim 1, wherein the motion lock hub comprises a member insertable into at least one of the longitudinal channel and the interior lumen and configured to frictionally engage the structure and a portion of the push-wire extension.

16. The catheter device of claim 15, wherein the member is wedged into the longitudinal channel to frictionally engage the push-wire extension and a portion of the structure.

17. The catheter device of claim 1, wherein the longitudinal channel comprises a clamp portion and wherein the motion lock hub comprises a control unit configured to adjust the clamp portion from an open position to a clamped position wherein the clamp portion frictionally engages the push-wire extension in the clamped position.

18. The catheter device of claim 1, wherein the motion lock hub is configured to selectively lock the push-wire extension and the proximal segment in fixed relation in conjunction with the connector module securing the distal segment to the proximal segment.

19. The catheter device of claim 1, wherein the connector module comprises a locking member disposed along a portion of the body and biased into a first secured position wherein the locking member engages a mating member disposed on or connected to the structure and wherein the push-wire extension is in operable connection with the locking member such that manipulation of the push-wire extension can cause the locking member to move away from the first secured position so that the distal segment and proximal segment are separated.

20. The catheter device of claim 1, wherein the longitudinal channel is disposed adjacent an outer surface of the structure through which the interior lumen extends.

21. (canceled)

22. (canceled)

23. (canceled)

24. (canceled)

25. (canceled)

26. (canceled)

27. (canceled)

28. (canceled)

29. (canceled)

30. (canceled)
